(19) United States
(12) Patent Application Publication
(10) Pub. No.: US 2025/0027074 A1
(43) Pub. Date: Jan. 23, 2025

(54) METHODS FOR CULTURING MICROORGANISMS IN A LIQUID CULTURE SYSTEM

(71) Applicants: Stephan Baehr, Scottsdale, AZ (US); Wei-Chin Ho, Tyler, TX (US); Michael Lynch, Mesa, AZ (US)

(72) Inventors: Stephan Baehr, Scottsdale, AZ (US); Wei-Chin Ho, Tyler, TX (US); Michael Lynch, Mesa, AZ (US)

(73) Assignee: Arizona Board of Regents on behalf of Arizona State University, Scottsdale, AZ (US)

(21) Appl. No.: 18/778,384

(22) Filed: Jul. 19, 2024

Related U.S. Application Data
(60) Provisional application No. 63/514,952, filed on Jul. 21, 2023.

(51) Int. Cl. C12N 15/10

(52) U.S. Cl. CPC C12N 15/1058

(57) ABSTRACT

The present invention provides a method of culturing microorganisms normally grown on a solid in liquid media, methods of use thereof to determine the presence or absence of the microorganism in a sample, and methods of use in a mutation accumulation assay.

### CROSS-REFERENCE TO RELATED APPLICATIONS

This application claims the benefit of the U.S. Provisional Application No. 63/514,952, filed Jul. 21, 2023, the disclosure of which is incorporated herein by reference in its entirety.

### STATEMENT REGARDING FEDERALLY SPONSORED RESEARCH OR DEVELOPMENT

This invention was made with government support under R35 GM122566 awarded by the National Institutes of Health, 2119963 awarded by the National Science Foundation, and W911NF-14-1-0411 awarded by DOD.DARPA. The government has certain rights in the invention.

### BACKGROUND OF THE INVENTION

Mutation rates describe the input of heritable variation into an organism. Because the average mutation is deleterious (A. Eyre-Walker, et al., Nature Reviews Genetics, (2007) 8(8):610-618), evolution pushes DNA mutation rates to the lower limits of natural selection (M. Lynch, et al., Nature Reviews Genetics, (2016) 17(11):704-714). DNA mutation events occur between 1 in 100,000,000 bases to 1 in 1,000,000,000,000 bases per cell division (M. Lynch, et al., Nature Reviews Genetics, (2016) 17(11):704-714), making their identification within sequenced genomes equivalent to the search for a needle in a rather large haystack. Techniques that amplify the signal of mutation events allow a general means of detection; the fluctuation test, first demonstrated in 1943 (S. E. Luria, et al., Genetics, (1943) 28(6):491-511; P. L. Foster, In DNA Repair, Part B, volume 409 of Methods in Enzymology, (2006) 195-213), uses a selective agent to make certain mutation events stand out, from which mutation rates can be estimated (D. E. Lea, et al., Journal of Genetics, (1949) 49(3):264-285; P. L. Foster, In DNA Repair, Part B, volume 409 of Methods in Enzymology, (2006) 195-213; Q. Zheng. New algorithms for Luria-Delbruck fluctuation analysis. Mathematical Biosciences, (2005) 196(2):198-214). With the advent of high-throughput sequencing, the mutation-accumulation (MA) experiment (M. Lynch, et al., Proceedings of the National Academy of Sciences, (2008) 105(27):9272-9277; M. Lynch, et al., Nature Reviews Genetics, (2016) 17(11):704-714), which employs recurrent single-cell bottlenecks typically on some surface like an agar plate, has become the gold standard for the empirical estimation of mutation rates. The analysis of a MA is straightforward, requiring the counting of new mutations arisen over a period of single-cell bottlenecks, and an estimation of the number of cell divisions, so as to come up with the numerator and denominator that comprises a mutation rate.

In microbiology, the MA has generally required growth on agar plates. This requirement reinforces a pervasive bias in microbiology, which typically requires bacteria to be growable on standard plates and in a timely manner, for humans to detect its existence at all. In contrast, metagenomic studies detect enormous diversity and cycling of microbes (JJ Lim, et al., Nature Communications (2023) 14(1)5682; Valles-Colomer et al., Nature (2023) 614(7946):125-135), many of which have never been cultured. Some microbes are known to grow quite slowly (Yoshida et al., Science (2016) 351(6278):1196-1199; Rodrigues-Oliveira et al., Nature (2023) 613(7943):332-339; Dabkowski et al., Connecticut Medicine (2013) 77(1):27-29); some do not grow readily on plates at all (Meixner et al., Bioengineering (2022) 9(4):178), and others inhabit biological niches that are hard to mimic on plates, for example the human urinary tract, which was once thought to be sterile (Bao et al., Annals of Translational Medicine (2017) 5:33-33). From the output of long term evolution experiments, to environmental isolates, and medical diagnoses of whether or not someone has a urinary tract infection regardless of what symptoms a patient may report, there is a general acknowledgement agar plates are not universally useful for scoring bacterial presence/growth.

Further specific criticism has been directed to MA experiments. An agar-based or other petri dish likely bears little resemblance to most cellular growth environments, from E. coli (S. Ishii, et al., Microbes and Environments, (2008) 23(2):101-108) to human cells. Oxygen is not a constant presence in many organism environments, though it is almost universally present in MA experiments unless expressly engineered out (S. Shewaramani, et al., PLOS Genetics, (2017) 13(1):1-22). Most cells used for MA experiments grow into colonies of some sort, a growth structure that is nutrient poor and perhaps different from an organism's natural growth structure, for example in a human gut where one might expect some level of nutrient flow or churn. Starvation has been proposed as a state that may be mutagenic (F. Taddei, et al., Molecular and General Genetics MGG, (1997) 256(3):277-281; P. L. Foster, Critical Reviews in Biochemistry and Molecular Biology, (2007) 42(5):373-397), though typically the time-frames are longer than 24 hours, and the experiments have trouble resolving the difference between higher mutation rates or more cell divisions occurring (S. Katz, et al., PLOS Genetics, (2013) 9(11):1-10; W. Wei, et al., Nature Communications, (2022) 13(1):4752).

Thus, there remains a need in the art for methods for determining mutation accumulation in conditions that more accurately reflect the environment of an organism. The present invention satisfies this unmet need.

### SUMMARY OF THE INVENTION

In one embodiment, the invention provides a method of culturing microorganisms or sample comprising cells comprising inoculating and incubating a microorganism or a sample comprising cells in a liquid culture system, wherein the liquid culture system comprises a vessel and a liquid medium.

In some embodiments, the cell is a cancerous cell, a diseased cell, or an atypical cell. In some embodiments, the cell is a HEK293 cell.

In some embodiments, the microorganism is one or more selected from the group consisting of bacteria, fungi, and yeast. In some embodiments, the microorganism is bacteria. In some embodiments, the bacteria is selected from the group consisting of: E. coli, Bacillus subtilis, Caulobacter crescentus, Mycobacterium tuberculosis, Streptococcus pneumoniae, Saphylococcus aureus, Listeria monocytogenes, Campylobacter, Yersinia pestis, Chlamydia trachomatis, Vibrio cholerae, and others. In some embodiments, the bacteria is from the genus synechocystis. In some embodiments, the microorganism is selected from the group consisting of Thermus aquaticus and Sulfolobus acidocaldarius.

In some embodiments, the microorganism or sample comprising cells is mismatch repair deficient.

In some embodiments, the liquid medium is one or more selected from the group consisting of Luria Bertani (LB) broth, Terrific Broth (TB), M9 minimal media, Sabouraud Dextrose (SD) broth, Yeast medium (YM) broth, (Ymin) yeast synthetic minimal media, BG-11 culture medium, a modified BG-11 culture medium, f/2 culture medium, a modified f/2 culture medium, ocean water, lake water, river water, wastewater, and other available water sources.

In some embodiments, the method further comprises at least one step of diluting the microorganism or sample comprising cells. In some embodiments, the microorganism or sample comprising cells is diluted at least twice. In some embodiments, the microorganism or sample comprising cells is diluted until a vessel comprises a single cell. In some embodiments, the vessel comprising a single cell is the vessel with the smallest turbidity, smallest color change, or a combination thereof. In some embodiments, the step of diluting the microorganism or sample comprising cells is automated.

In some embodiments, the microorganism or sample comprising cells is incubated at a temperature of about 25° C. to about 70° C. In some embodiments, the medium has a pH of 3 to 10.

In some embodiments, the method further comprises an analysis step. In some embodiments, the analysis step is selected from the group consisting of: plating the microorganism or the sample comprising cells on a media, differential staining, antibody staining, and high-throughput sequencing. In some embodiments, the media comprises permissive growth conditions or inhibitory growth conditions. In some embodiments, the differential staining is Gram staining. In some embodiments, the antibody staining is ELISA. In some embodiments, the high-throughput sequencing is metagenomic sequencing.

In some embodiments, the invention provides an assay system for measuring mutation accumulation in a microorganism or a sample comprising cells comprising the method of the invention and a DNA analysis step. In some embodiments, the DNA analysis step is DNA sequencing.

In some embodiments, the invention provides an assay system for detecting the presence of a microorganism or a sample comprising cells in a sample comprising the method of the invention. In some embodiments, the assay system is used to detect urinary tract infection.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a schematic of the experimental design. From a single colony streaked on a plate, liquid and plate MA's were started in tandem. The plate MA used standard methods. The liquid MA begins with a serial dilution to the point of getting 0 or 1 cells in a well. Three different serial dilutions are used to ensure sufficient dilution.

FIG. 2 depicts a representative quantification of mutation rate estimation. Multiple estimates of the E. coli MMR—mutation rate are consistent over time. The liquid and plate experiments of this report produced mutation rate estimates more similar to each other than the difference between plate MA experiments over time. Error bars reflect the standard error of the mean.

FIG. 3A and FIG. 3B depict a representative quantification of mutation spectra of MMR—E. coli. FIG. 3A depicts a representative quantification demonstrating that across MA experiments, the mutation spectrum ratios of MMR—E. coli mutations holds relatively constant, particularly in the case of the plate MA. FIG. 3B depicts a representative quantification of mutation rates rather than mutation ratios. In this graph, the mutations from hypermutator line P35 are omitted. Error bars reflect poisson 95% confidence intervals.

FIG. 4A and FIG. 4B depict a representative quantification of cell counts per day for MMR—E. coli. FIG. 4A depicts a representative CFU estimate of the number of cells present in a culture after 24 hours of growth, from resuspended colonies or from 1 mL liquid cultures for the plate and liquid MA's, respectively. FIG. 4B depicts a representative quantification of the coefficient of variation (CV) summed over the two MA's, indicating that there is a greater CV and distribution of CV in the plate MA than the liquid MA.

FIG. 5A through FIG. 5D depict a representative quantification of 96-well vs. CFU Fitness, Carrying Capacity vs Ancestor in MMR—E. coli. FIG. 5A depicts a representative quantification of an example growth curve from a plate reader in a single well, in which culture turbidity is measured at 15 min. The red line indicates the calculation of fitness, the blue line demonstrates how carrying capacity is calculated. FIG. 5B depicts a representative quantification of the relative fitness, normalized to ancestral fitness. FIG. 5C depicts a representative quantification of the relative carrying capacity at 12 hr, normalized to ancestral carrying capacity. FIG. 5D depicts a representative quantification of cell count at 12 hr as estimated by CFU.

FIG. 6A and FIG. 6B depict representative experimental results demonstrating a decline in 24 hr cell count of liquid MA line 02. FIG. 6A depicts a representative quantification of 24 hr CFU estimates of cell count in a time series of line liquid #2 (L02), with a focus on the drop between day 14 and day 15. FIG. 6B depicts a table summarizing four mutations were fixed between day 14 and 15.

FIG. 7A and FIG. 7B depict representative images of a serial dilution strategy.

FIG. 7A depicts a schematic of a serial dilution strategy. FIG. 7B depicts a representative image of a plate using the serial dilution strategy.

### DETAILED DESCRIPTION

The present invention relates to methods of culturing microorganisms in liquid culture. Experiments requiring single-cell bottlenecks have typically been done on agar plates which reinforces a bias of requiring microorganisms, particularly bacteria, to grow on standard plates and in a timely manner for human detection. Some microbes are known to grow quite; some do not grow readily on plates at all, and others inhabit biological niches that are hard to mimic on plates, for example the human urinary tract, which was once thought to be sterile.

The present invention is based, in part, on the discovery that single-cell bottlenecks can be generated through liquid culture. The liquid culture bottlenecks are similar to agar plate bottlenecks. Liquid mutation accumulation (MA) experiments recapitulate the mutation rate estimated for MMR—E. coli in liquid LB culture vs. plate LB culture. Therefore, the present invention is further based, in part, on the discovery that microorganisms that are normally plated can be cultured in liquid media.

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of ±20%, ±10%, ±5%, ±1%, or ±0.1% from the specified value, as such variations are appropriate to perform the disclosed methods. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or there below. Furthermore, the phrases “less than about [a value]” or “greater than about [a value]” should be understood in view of the definition of the term “about” provided herein.

As used herein, the term “biomass” is intended to mean the collection of biological matter, made up of cells, that results from the culturing process of a microorganism under suitable conditions for the growth of that organism in culture. In some cases, the biomass includes simply the cells and their contents and in some cases, the biomass includes additionally any macromolecules, such as proteins, that are secreted into the culture, outside the boundary of the cell membrane.

As used herein, the term “carbon source” is intended to mean a raw material input to an industrial process that contains carbon atoms that can be used by the microorganisms in a culture. For example, industrial cultures of microorganisms may use glucose as a source of carbon atoms. As provided herein, in addition to typical carbon sources such as sugars and amino acids. In some cases, a culture is grown in a medium containing a single usable compound that contains carbon atoms. As carbon is an element that is essential for life, the culture must have, in this example, metabolic pathways for converting the single compound containing carbon atoms into many other biological molecules necessary for the organism's survival.

As used herein, the term “culturing” is intended to mean the growth or maintenance of microorganisms under laboratory or industrial conditions. The culturing of microorganisms is a standard practice in the field of microbiology. Microorganisms can be cultured using liquid or solid media as a source of nutrients for the microorganisms. In addition, some microorganisms can be cultured in defined media, in which the liquid or solid media are generated by preparation using purified chemical components. The composition of the culture media can be adjusted to suit the microorganism or the industrial purpose for the culture.

As used herein, the term “endogenous polynucleotides” is intended to mean polynucleotides derived from naturally occurring polynucleotides in a given organism. The term “endogenous” refers to a referenced molecule or activity that is present in the host. Similarly, the term when used in reference to expression of an encoding nucleic acid or polynucleotide it refers to expression of the encoding nucleic acid or polynucleotide contained within the microbial organism.

As used herein, the term “exogenous polynucleotides” is intended to mean polynucleotides that are not derived from naturally occurring polynucleotides in a given organism. Exogenous polynucleotides may be derived from polynucleotides present in a different organism. The exogenous polynucleotides can be introduced into the organism by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid. As set forth in the invention a nucleic acid need not include all of its relevant or even complete coding regions on a single polymer and the invention provided herein contemplates having complete or partial coding regions on different polymers.

The term “infection” means an initial or primary (acute) or a chronic infection. An infection may be “infectious” in the sense that other sites in the infected host subject, or contagious to other subjects (cross-infection), or may be latent. An initial/primary (acute) infection can cause mild, moderate or severe pathogenesis or symptoms, or be asymptomatic. A primary/initial infection may or may not be self-limiting, and can become progressively worse, or become latent. A “latent” infection in a host subject is a state in which the infection (e.g., virus) evades immune clearance and remains in the host subject, which infection can be chronic, even lifelong. In the latent state illness or symptoms may not be present or may be mild. Reactivation of an infection means activation in the host subject following a period of latency. Reactivation is associated with increased replication and proliferation in a subject. Symptoms and pathologies associated with or caused by reactivation may also increase.

The term “bacterial infection” or “bacterial infectious disease” generally refers to all types of diseases caused by bacteria or bacteria-derived toxins. In general, the bacteria or bacteria-derived toxins breach defense systems of a host to cause infectious diseases. In this case, representative examples of the infectious diseases include pneumonia and pulmonary tuberculosis occurring in the lungs; osteomyelitis and osteoarthritis occurring in bone joints; infective endocarditis occurring in the heart; encephalitis and meningitis occurring in the brain; nephritis occurring in the kidneys; systemically occurring sepsis, etc.

The term “serious bacterial infection” or “serious bacterial infectious disease” refers to the expression of serious bacterial infections including cases in which diseases such as pneumonia, sepsis, infective endocarditis, nephritis, osteoarthritis, and the like have a high mortality rate or are difficult to heal due to bacterial infections.

As used herein, the terms “microbe”, “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. Microorganism types include without limitation, bacteria (e.g., Mycoplasma, coccus, Bacillus, Rickettsia, spirillum), fungi (e.g., filamentous fungi, yeast), nematodes, protozoans, archaea, algae, dinoflagellates, viruses (e.g., bacteriophages), viroids and/or a combination thereof. Organism strains are subtaxons of organism types, and can be for example, a species, sub-species, subtype, genetic variant, pathovar or serovar of a particular microorganism. The term also includes cell cultures of any species that can be cultured for the production of a biochemical.

As used herein, “isolate,” “isolated,” “isolated microbe,” and like terms, are intended to mean that the one or more microorganisms has been separated from at least one of the materials with which it is associated in a particular environment (for example soil, water, animal tissue). Thus, an “isolated microbe” does not exist in its naturally occurring environment; rather, it is through the various techniques described herein that the microbe has been removed from its natural setting and placed into a non-naturally occurring state of existence. Thus, the isolated strain may exist as, for example, a biologically pure culture, or as spores (or other forms of the strain) in association with an acceptable carrier.

As used herein, “microbial ensemble” refers to a composition comprising one or more active microbes identified by methods, systems, and/or apparatuses of the present disclosure and that does not naturally exist in a naturally occurring environment and/or at ratios or amounts that do not exist in a nature. For example, a microbial ensemble (also synthetic ensemble or bioensemble) or aggregate could be formed from one or more isolated microbe strains, along with an appropriate medium or carrier. Microbial ensembles can be applied or administered to a target, such as a target environment, population, individual, animal, and/or the like.

The terms “microbiological culture”, “microbial culture”, or “microorganism culture” refer to a method or system for multiplying microorganisms through reproduction in a predetermined culture medium, including under controlled laboratory conditions.

Microbiological cultures, microbial cultures, and microorganism cultures are used to multiply the organism, to determine the type of organism, or the abundance of the organism in the sample being tested. In liquid culture medium, the term microbiological, microbial, or microorganism culture generally refers to the entire liquid medium and the microorganisms in the liquid medium regardless of the vessel in which the culture resides. A liquid medium is often referred to as “media”, “culture medium”, or “culture media”. The act of culturing is generally referred to as “culturing microorganisms” when emphasis is on plural microorganisms. The act of culturing is generally referred to as “culturing a microorganism” when importance is placed on a species or genus of microorganism. Microorganism culture is used synonymously with culture of microorganisms.

The term “inoculate” refers to implanting or introducing microorganisms into a culture medium. Inoculate or inoculating a culture of microorganisms in the described culture conditions throughout the specification refers to starting a culture of microorganisms in the culture conditions, as is commonly used in the art of microorganism culturing. The microorganisms that are introduced into a culture medium may be referred to as seed or inoculum.

The terms “subject,” “patient,” “individual,” and the like are used interchangeably herein, and refer to any animal, or cells thereof whether in vitro or in situ, amenable to the methods described herein. In some non-limiting embodiments, the patient, subject or individual is a mammal, bird, poultry, cattle, pig, horse, sheep, ferret, primate, dog, cat, guinea pig, rabbit, bat, or human. In one embodiment, the patient, subject, or individual is a human.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Single-Cell Bottlenecking by Liquid Culture

The present invention is based, in part, on the discovery that microorganisms that are normally plated can be cultured in liquid media.

### Liquid Single Cell Bottlenecking

According to the invention, a sample comprising microorganisms is collected. The sample is sequentially diluted until a single cell of the microorganism is in a vessel. The vessel comprising the single cell of the microorganism is then incubated such that the microorganism is maintained and/or grown.

In some embodiments, the sample does not comprise microorganisms. In some embodiments, the sample comprises eukaryotic cells. In some embodiments, the sample comprises cells from multicellular organisms.

In some embodiments, the sample is diluted between 1:1 and 1:2. In some embodiments, the sample is diluted between 1:2 and 1:3. In some embodiments, the sample is diluted between 1:3 and 1:4. In some embodiments, the sample is diluted between 1:4 and 1:5. In some embodiments, the sample is diluted between 1:5 and 1:10. In some embodiments, the sample is diluted between 1:10 and 1:100. In some embodiments, the sample is diluted between 1:100 and 1:1,000. In some embodiments, the sample is diluted between 1:1,000 and 1:10⁶ (1,000,000). In some embodiments, the sample is diluted between 1:10⁶ and 1:10⁹. In some embodiments, the sample is diluted between 1:10⁹ and 1:10¹². In some embodiments, the sample is diluted between 1:10¹² and 1:10¹⁵.

In some embodiments, the sample is diluted at the same ratio the first time and the subsequent time(s). In some embodiments, the sample is diluted at different ratio the first and the subsequent time(s).

In some embodiments, the sample is not diluted. In some embodiments, the sample is diluted 1, 2, 3, 4, 5, 6, 7, 8, 9, or at least 10 times. In some embodiments, the sample is sequentially diluted until there is a single cell. In some embodiments, the sample is sequentially diluted until there are 10 or less cells. In some embodiments, the sample is sequentially diluted until there are 100 or less cells. In some embodiments, the sample is diluted until there are 1,000 or less cells. In some embodiments, the sample is diluted until there are 10,000 or less cells. In some embodiments, the sample is diluted until there are 100,000 or less cells. In some embodiments, the sample is diluted until there are 1,000,000 or less cells.

In some embodiments, the sample is maintained and/or grown between dilution steps. In some embodiments, the sample is maintained and/or grown by incubating the sample. In some embodiments, the sample that comprises a single cell is the sample that results in a media with low turbidity, color change, or a combination thereof after a plurality of sequential dilution steps and maintenance and/or growth steps.

In some embodiments, the sample that comprises a single cell is the sample that results in a media with low turbidity after a plurality of sequential dilution steps and maintenance and/or growth steps. In some embodiments, the media with low turbidity has the lowest turbidity compared to other samples that are sequentially diluted. In some embodiments, the sample with the lowest turbidity is the sample with the single cell. In some embodiments, the sample with the lowest turbidity is the sample with 10 or less cells. In some embodiments, the sample with the lowest turbidity is the sample with 100 or less cells. In some embodiments, the sample with the lowest turbidity is the sample with 10³ or less cells, 10⁴ or less cells, 10⁵ or less cells, or 10⁶ or less cells. In some embodiments, 2, 3, 4, 5, 6, 7, 8, 9, or 10 or more samples comprise media with similar turbidities. In some embodiments, the 2, 3, 4, 5, 6, 7, 8, 9, or 10 or more samples that comprise media with similar turbidities have the lowest turbidity compared to another sample.

In some embodiments, the sample that comprises a single cell is the sample that results in a media with a color change after a plurality of sequential dilution steps and maintenance and/or growth steps. In some embodiments, the media with color change has the smallest color change compared to other samples that are sequentially diluted. In some embodiments, the sample with the smallest color change is the sample with the single cell. In some embodiments, the sample with the smallest color change is the sample with 10 or less cells. In some embodiments, the sample with the smallest color change is the sample with 100 or less cells. In some embodiments, the sample with the smallest color change is the sample with 10³ or less cells, 10⁴ or less cells, 10⁵ or less cells, or 10⁶ or less cells. In some embodiments, 2, 3, 4, 5, 6, 7, 8, 9, or 10 or more samples comprise media with similar colors. In some embodiments, the 2, 3, 4, 5, 6, 7, 8, 9, or 10 or more samples that comprise media with similar colors have the smallest color change compared to another sample.

In some embodiments, the media with color change has the smallest color change compared to other samples that are sequentially diluted. In some embodiments, the sample with the largest color change is the sample with the single cell. In some embodiments, the sample with the largest color change is the sample with 10 or less cells. In some embodiments, the sample with the largest color change is the sample with 100 or less cells. In some embodiments, the sample with the largest color change is the sample with 10³ or less cells, 10⁴ or less cells, 10⁵ or less cells, or 10⁶ or less cells. In some embodiments, 2, 3, 4, 5, 6, 7, 8, 9, or 10 or more samples comprise media with similar colors. In some embodiments, the 2, 3, 4, 5, 6, 7, 8, 9, or 10 or more samples that comprise media with similar colors have the largest color change compared to another sample.

In some embodiments, the method of diluting the sample is done by a device. In some embodiments, the device is an automated device. In some embodiments, the automated device is a liquid handling machine. In some embodiments, the automated device processes the sample. For example, the system may be configured to automate all of the steps involved in diluting the sample, including but not limited to, de-capping/capping of sample vials and/or tubes, pipetting, adding media to a vessel, and inoculating the sample in the vessel with the media. The system may further repeat these steps as necessary as described in the disclosure. In some embodiments, the automated device comprises an additional system and/or module for incubating the sample. Appropriate incubation conditions are described later in the disclosure. The automated device may incubate the samples according to these conditions.

The method of the present invention results in the growth or maintenance of the microorganism. The liquid culture comprising the microorganism may be used for any downstream process known by those of ordinary skill in the art. These downstream processes include but are not limited to maintenance of a microorganism, growth of a microorganism, a mutation accumulation assay, diagnosis of a sample, diagnosis of a disease, measuring the mutagenic potential of various chemicals to which living things may be exposed, and measuring chromatin mutation rates.

An illustrative example of chemicals include but is not limited to chemicals considered to be carcinogenic. The method of the present invention provides an alternative metric as a means for scoring the mutagenic potential of a chemical or a substance that is exposed to various cell types.

Methods of measuring chromatin mutation rates are known in the art which may include but is not limited to measuring DNA methylation mutation rates per cell division, measuring histone methylation per cell division, measuring histone acetylation per cell division, and measuring any other histone or DNA modification per cell division.

### Sample Preparation

Test samples may be obtained from an individual, subject, or patient. Methods of obtaining test samples are well-known to those of skill in the art. Samples may include, but are not limited to, saliva, whole blood, serum, plasma, amniotic fluid, cervical secretions, vaginal secretions, cerebrospinal fluid (CSF), pericardial fluid, pleural fluid, synovial fluid, urine, eye fluid, blood, feces, sweat, bile, serum, plasma, tissue biopsy, and the like. For example, in one embodiment, the sample is one or more selected from the group consisting of: saliva, whole blood, serum, plasma, amniotic fluid, cervical secretions, vaginal secretions, cerebrospinal fluid (CSF), pericardial fluid, pleural fluid, synovial fluid, urine, eye fluid, blood, feces, sweat, bile, serum, plasma, and tissue biopsy.

In one aspect, the present invention relates to a method of preparing a sample. In one embodiment, the method comprises providing a biological sample obtained from the subject. The biological sample can be a sample from any source, such as a body fluid (e.g., saliva, blood, plasma, serum, synovial fluid, cervical secretions, vaginal secretions, urine, amniotic fluid, etc.), or a tissue, or an exosome, or a cell, or a combination thereof. A biological sample can be obtained by appropriate methods, such as, by way of examples, biopsy or fluid draw.

Samples may also include non-biological samples including but not limited to ocean water, lake water, river water, wastewater, or other available water sources. In one embodiment, the sample is one or more selected from the group consisting of ocean water, lake water, river water, wastewater, and another available water source.

Samples may also include solid materials, such as biological tissue, soil, rock, meat, vegetables, fruit, and the like. In certain embodiments, solid samples are dissolved or resuspended in an aqueous solution to obtain a liquid sample.

Samples may also include tissues, primary cells, cell lines, or organoids from multicellular organisms.

### Microorganisms

The examples describe specific modifications and evaluations to certain bacterial microorganisms. The scope of the invention is not meant to be limited to such species, but to be generally applicable to a wide range of suitable microorganisms. Generally, a microorganism used for the present invention may be selected from bacteria, cyanobacteria, filamentous fungi and yeasts. As the genomes of various species become known, the present invention easily may be applied to an ever-increasing range of suitable microorganisms. Further, given the relatively low cost of genetic sequencing, the genetic sequence of a species of interest may readily be determined to make application of aspects of the present invention more readily obtainable (based on the ease of application of genetic modifications to a microorganism having a known genomic sequence).

In some embodiments, the microorganism is one or more selected from the group consisting of: Bacillus subtilis, Caulobacter crescentus, Mycobacterium tuberculosis, Streptococcus pneumoniae, Saphylococcus aureus, Listeria monocytogenes, Campylobacter, Yersinia pestis, Chlamydia trachomatis, Vibrio cholerae, and others.

In some embodiments, the microorganism is an ecological bacteria. In some embodiments, the microorganism is from the genus synechocystis.

In some embodiments, the microorganism is an archaeal organisms. In some embodiments, the microorganism is an extremophile. In some embodiments, the microorganism is selected from the group consisting of: Thermus aquaticus and Sulfolobus acidocaldarius.

In some embodiments, the microorganism is a Eukaryote. In some embodiments, the Eukaryote is single-celled. In some embodiments, the microorganism is Saccharomyces cerevisiae. In some embodiments, the microorganism is selected from the group consisting of yeasts, ciliates, and euglenids.

It is further appreciated, in view of the disclosure, that any of the above microorganisms may be a modified microorganism or a recombinant microorganism. The ability to genetically modify the host is essential for the production of any recombinant microorganism. In some embodiments, the microorganism is genetically modified. The mode of gene transfer technology may be by electroporation, conjugation, transduction or natural transformation. A broad range of host conjugative plasmids and drug resistance markers are available. The cloning vectors are tailored to the host microorganisms based on the nature of antibiotic resistance markers that can function in that host.

In some embodiments, the microorganism is modified to be mismatch repair deficient.

### Other Cells

Cells used in the present invention need not be a microorganism. Any sample comprising cells may be used in the present invention. In some embodiments, the cell is from a cell line derived from humans or another multicellular organism. In some embodiments, the cell line derived from humans or another multicellular organism can tolerate serial dilution. In some embodiments, the cell line is derived from cancerous tissues. In some embodiments, the cell line is derived from a diseased tissue. In some embodiments, the cell line is derived from an atypical tissue. In some embodiments, the cell line is HEK293 cells.

### Media and Culture Conditions

Another aspect of the invention regards media and culture conditions that comprise microorganism and genetically modified microorganisms of the invention and optionally supplements. Liquid media comprises suitable minerals, salts, cofactors, buffers and other components, known to those skilled in the art, suitable for the growth of the cultures.

Suitable growth media in the present invention are common commercially prepared media, including but not limited to Luria Bertani (LB) broth, Terrific Broth (TB), M9 minimal media, Sabouraud Dextrose (SD) broth. Yeast medium (YM) broth, (Ymin) yeast synthetic minimal media, and minimal media as described herein, such as M9 minimal media. Other defined or synthetic growth media may also be used, and the appropriate medium for growth of the particular microorganism will be known by one skilled in the art of microbiology.

The microorganisms are inoculated in an aqueous culture medium contained in any suitable vessel for growth. In some embodiments, the culture medium may be any liquid culture medium suitable for culturing microorganisms, including but not limited to a BG-11 culture medium, a modified BG-11 culture medium, an f/2 culture medium, and a modified f/2 culture medium. In some embodiments, the culture medium comprises any one or more of: ocean water, lake water, river water, wastewater, or other available water sources; available water sources cleaned via filtration or sterilization before inoculation with a microorganism culture; and an aqueous media inoculated with beneficial microbes (e.g., bacteria) to jumpstart the microorganism culture. In some embodiments, parameters of a culture may be manipulated and beneficial microbes (e.g., bacteria) may be added to the culture media as needed depending on microorganisms present in the culture and health of the culture.

In various embodiments a minimal media may be developed and used that does not comprise, or that has a low level of addition of various components, for example less than 10, 5, 2 or 1 g/L of a complex nitrogen source including but not limited to yeast extract, peptone, tryptone, soy flour, corn steep liquor, or casein. These minimal medias may also have limited supplementation of vitamin mixtures including biotin, vitamin B12 and derivatives of vitamin B12, thiamin, pantothenate and other vitamins. Minimal media may also have limited simple inorganic nutrient sources containing less than 28, 17, or 2.5 mM phosphate, less than 25 or 4 mM sulfate, and less than 130 or 50 mM total nitrogen.

Liquid media, which is used in embodiments of the present invention with microorganisms or genetically modified microorganisms, must contain suitable carbon substrates. Carbon sources may include, but are not limited to, monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Additionally, the carbon source may also be one-carbon substrates such as carbon dioxide, carbon monoxide, or methanol for which metabolic conversion into key biochemical intermediates has been demonstrated. Additionally, the carbon substrate may also be carbon dioxide and hydrogen or a combination thereof, such as syngas. In addition to one and two carbon substrates methylotrophic microorganisms are also known to utilize a number of other carbon containing compounds such as methylamine, glucosamine and a variety of amino acids for metabolic activity. Other suitable sources include xylose, arabinose, other cellulose-based C-5 sugars, high-fructose corn syrup, and various other sugars and sugar mixtures as are available commercially. Sucrose may be obtained from feedstocks such as sugar cane, sugar beets, cassava, bananas or other fruit, and sweet sorghum. Glucose and dextrose may be obtained through saccharification of starch-based feedstocks including grains such as corn, wheat, rye, barley, and oats. Also, in some embodiments, all or a portion of the carbon source may be glycerol. Alternatively, glycerol may be excluded as an added carbon source. In some embodiments, a mixture of the carbon sources may be used.

In one embodiment, the carbon source is selected from glucose, fructose, sucrose, dextrose, lactose, glycerol, and mixtures thereof. Variously, the amount of these components in the carbon source may be greater than about 50%, greater than about 60%, greater than about 70%, greater than about 80%, greater than about 90%, or more, up to 100% or essentially 100% of the carbon source.

In addition, methylotrophic microorganisms are known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth C1 Compd. (Int. Symp.), 7th (1993), 415-32. Editor(s): Murrell, J. Collin; Kelly, Don P. Publisher: Intercept, Andover. UK). Similarly, various species of Candida will metabolize alanine or oleic acid (Sulter et al., Arch. Microbiol. 153:485-489 (1990)). Hence it is contemplated that the source of carbon utilized in embodiments of the present invention may encompass a wide variety of carbon-containing substrates.

In addition, fermentable sugars may be obtained from cellulosic and lignocellulosic biomass through processes of pretreatment and saccharification, as described, for example, in U.S. Patent Publication No. 2007/0031918A1, which is herein incorporated by reference. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure. Any such biomass may be used to provide a carbon source. Various approaches to breaking down cellulosic biomass to mixtures of more available and utilizable carbon molecules, including sugars, include: heating in the presence of concentrated or dilute acid (e.g., <1% sulfuric acid); treating with ammonia; treatment with ionic salts; enzymatic degradation; and combinations of these. These methods normally follow mechanical separation and milling, and are followed by appropriate separation processes.

Typically, cells are grown at a temperature in the range of about 25° C. to about 40° C. in an appropriate medium, as well as up to 70° C. for thermophilic microorganisms. Suitable pH ranges for the culture are between pH 3.0 to pH 10.0, where pH 6.0 to pH 8.0 is a typical pH range for the initial condition. However, the actual culture conditions for a particular embodiment are not meant to be limited by these pH ranges.

Culture may be performed under aerobic, microaerobic, or anaerobic conditions, with or without agitation.

### Detection of Microorganism

The liquid culture comprising the microorganism may be analyzed for the presence or absence or identity of a microorganism.

Techniques to detect, identify, and/or analyze microorganisms are known in the art. Non-limiting examples include but are not limited to plating microorganisms, such as bacteria, on different media types to see permissive and inhibitory growth conditions. Another method involves differential staining of microorganisms, such as bacteria, with different chemicals such as Gram staining. A third method involves antibody staining to look for species-identifying proteins, for example, by ELISA detection protocols. A fourth method involves metagenomic sequencing, a variant of high-throughput sequencing which blasts reads to all known samples.

In one embodiment, the liquid culture comprising the microorganism is analyzed by plating the microorganism on a media. In some embodiments, the media the microorganism is plated on comprises permissive growth conditions. In some embodiments, the media the microorganism is plated on comprises inhibitory growth conditions. In some embodiments, the liquid culture comprising the microorganism is analyzed by differential staining. In some embodiments, the differential staining is performed with chemicals. In some embodiments, the chemical is Gram staining. In some embodiments, the liquid culture comprising the microorganism is analyzed by identification of proteins. In some embodiments, the identification of proteins is done by ELISA. In some embodiments, the liquid culture comprising the microorganism is analyzed by high-throughput sequencing. In some embodiments, the liquid culture comprising the microorganism is analyzed by metagenomic sequencing.

In one embodiment, nucleic acid from a liquid culture may be isolated and analyzed by any suitable technique to identify the microorganism. Exemplary methods for analysis of nucleic acids include, but are not limited to, amplification techniques, such as PCR and RT-PCR (including quantitative variants), and hybridization techniques, such as in situ hybridization, microarrays, and blots. In one embodiment, the nucleic acid may be analyzed to identify signature sequences from the microorganism of interest. The nucleic acid may be analyzed by PCR using primers that anneal, allow amplification, specifically to a signature nucleic acid sequence that occurs in the target microorganism.

The nucleic acid may be analyzed by PCR using primers that anneal specifically to a signature nucleic acid sequence that occurs in the target microorganism. The primers may anneal specifically to the signature nucleic acid sequence and/or may allow amplification of the specific signature nucleic acid. To increase the specificity more than one, more than two, more than three, more than four, more than five, more than six, more seven or more than eight signature sequences may be considered for the target microorganism to be detected. In one embodiment, at least 1, 2, 3, 4, 5, 6, 7, 8, 9, 10, 15, 20, 25, 30, 35, 40, 45, 50, 55, 60, 65, 70, 75, 80, 85, 90, 95, 100, or more than 100 signature species for at least one microorganism are evaluated in a single assay. Exemplary assays that can be used to evaluate multiple signature sequences, include, but are not limited to, microarrays, and q-PCR.

In one embodiment, the liquid culture comprising the microorganism is analyzed by sequencing. The nucleic acid sequence may be analyzed by sequencing at least a portion of the genomic DNA or RNA. Methods for performing whole or partial genome sequencing are known in the art and include, but are not limited to, exome sequencing, whole genome sequencing, and 16S rRNA sequencing. In various embodiments, sequencing may be done through Sanger sequencing, or through high-throughput next-generation sequencing techniques (e.g., using an Illumina based Hi-Seq, or Mi-Seq or Life Technologies PGM based sequencing platform).

### Diagnosis

The detection of the presence or absence of a microorganism may be used to diagnose a subject. Therefore, the method of the present invention may be used in an assay to diagnose a subject for the presence or absence of a disease or infection associated with the microorganism. In one embodiment, the disease or disorder is urinary tract infection (UTI).

In one embodiment, the method comprises detecting one or more microorganism in at least one biological sample of the subject. In various embodiments, the level of one or more of microorganisms of the invention in the biological sample of the subject is compared with the level of a corresponding biomarker in a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of.

In one embodiment, the method comprises detecting one or more microorganisms simultaneously in two or more different biological samples of the subject. In one embodiment, the method comprises detecting one or more microorganisms sequentially in two or more different biological samples of the subject.

In various embodiments, the level of one or more of microorganisms of the invention in each biological sample of the subject is compared with the level of a corresponding biomarker in an appropriate comparator. In one embodiment, a subject is diagnosed as having or being at risk of a disease or disorder when the level of the microorganisms in each of two or more biological samples is increased or decreased relative to the appropriate comparator.

In one embodiment, the method of the invention allows detection of the microorganism at very early stages of infection, and before any clinical symptoms are visible. The method allows identification of the presence of live microorganisms when they are present in a sample at very low numbers, for example less than 10 cells per sample.

In one embodiment, the method of the invention is used to monitor the efficacy of a treatment, and to screen for whether the numbers of microorganism are reducing as treatment is given.

### Liquid Mutation Accumulation (MA) Assay

The liquid culture comprising the microorganism may be analyzed for changes between microorganisms in the initial culture and microorganisms in subsequent cultures. Illustrative examples of changes that may be analyzed include but are not limited to growth rate, resistance to stress (antibiotic stress, temperature stress, pH stress, etc), nucleic acid mutations (DNA, RNA, etc).

In one embodiment, the microorganism is analyzed for changes between an initial culture and a subsequent culture. In one embodiment, the microorganism is analyzed for mutations. In one embodiment, the microorganism is analyzed for mutation rates. Mutations and mutation rates can be analyzed using any technique known in the art.

As a non-limiting, illustrative example, the analysis of a mutation accumulation may be done by counting new mutations that arise over a period of single-cell bottlenecks and dividing it by the number of cell divisions. For example, from a 10 mL liquid LB tube, an ancestor line frozen stock was made and then 16 wells of 20 μL were taken and run through serial dilution. E. coli were serially diluted to the −5 and −6 wells by standard methods and multichannel pipettors in 180 μL 1×PBS in 96 well plates. Then, 100 μL of the −5 was added to 900 μL of 1×PBS to create a “−7” dilution of 1 mL per sample, and the same was done to the −6 well to create a “−8” dilution of 1 mL per sample.

From the −7 dilution, 20 μL was inoculated to a single row of a 96-deepwell plate holding 950 μL of liquid LB, 8 wells. From the −8 dilution, a second row for the same sample was loaded with 50 μL, and for a 3rd row the −8 dilution was again used to inoculate 20 μL.

In one embodiment, this results in an order of magnitude scale of dilution in the 96 deepwell plate, where each liquid sample comprises now 24 replicates of varying serial dilution. On a 96 deepwell plate four samples were arrayed, for a total of 4 96 deepwell plates, or 16 replicates, per transfer day. After 24 hours, some of the serial dilution wells have turbid growth, while others remain clear. At the lower serial dilutions, a majority of the wells should be clear. As a rule, the turbid well from the lowest serial dilution would be chosen for the next round of serial dilution. In the event of a tie, the one on the left-hand side was chosen.

After 15 days, frozen stocks were made of both the liquid and the plate MA. Based on the previous MA experiment, it was expected 20 days would result in a sufficient number of mutations to analyze the data.

In one embodiment, after the MA's were complete and frozen stocks obtained, DNA was extracted by first growing the frozen stocks overnight in 10 mL tubes and then running the Promega Wizard DNA Extraction Kit, with minor modifications immaterial to results save by increasing yield to >1 μg per sample. Genomic DNA was quantified, and submitted for library prep and sequencing.

In principle, single-cell bottleneck can be obtained in several ways. One can streak bacteria on a plate until distinct colonies are acquired, or, as proposed and demonstrated here, serial dilution may be employed to the point of getting 0 or 1 cell in an inoculum used to stimulate growth (or not) in a well of growth media.

The present disclosure also contemplates an analysis of mutations in modified microorganism. Microorganisms used in the mutation accumulation assay may be genetically modified by techniques known in the art including but not limited to transformation, electroporation, conjugation, and/or transduction. Sequences, mutations, and mutation rates may then be compared between the microorganism and the modified microorganism.

The present disclosure also contemplates an analysis of mutations in microorganisms as a result of changes in media or culture conditions. The media may be modified as a result of changes in carbon source, concentration of the carbon source, pH, salt, nitrogen source, vitamins, etc. The culture conditions may be modified as a result of changes in temperature, agitation, gas conditions, etc. Sequences, mutations, and mutation rates in the microorganism may then be compared between microorganisms growing in the media and the modified media. Sequences, mutations, and mutation rates in the microorganism may also be compared between microorganisms growing in the culture conditions and the modified culture conditions.

### Kits

In some embodiments, the present disclosure provides a kit for culturing microorganisms or sample comprising cells comprising inoculating and incubating a microorganism or a sample comprising cells in a liquid culture system, wherein the liquid culture system comprises a vessel and a liquid medium. In some embodiments, the liquid medium is one or more selected from the group consisting of Luria Bertani (LB) broth, Terrific Broth (TB), M9 minimal media, Sabouraud Dextrose (SD) broth, Yeast medium (YM) broth, (Ymin) yeast synthetic minimal media, BG-11 culture medium, a modified BG-11 culture medium, f/2 culture medium, a modified f/2 culture medium, ocean water, lake water, river water, wastewater, and other available water sources. In some embodiments, the medium has a pH of 3 to 10.

In some embodiments, the kit further comprises compositions for analysis of the microorganisms or sample comprising cells.

In some embodiments, the analysis is plating the microorganism or the sample comprising cells on a media. In some embodiments, the media comprises permissive growth conditions or inhibitory growth conditions. Thus, in some embodiments, the kit further comprises factors to be added to the media that permits or inhibits growth of the microorganisms or sample comprising cells.

In some embodiments, the analysis is differential staining. In some embodiments, the differential staining is Gram staining. Thus, in some embodiments, the kit further comprises a primary stain, a mordant, a decolorizer, a counterstain, or a combination thereof. In some embodiments, the primary stain is crystal violet. In some embodiments, the mordant is Gram's iodine solution. In some embodiments, the decolorizer comprises acetone, an alcohol, or a combination thereof. In some embodiments, the alcohol is ethanol. In some embodiments, the counterstain is fuchsin solution.

In some embodiments, the analysis is antibody staining. In some embodiments, the antibody staining is ELISA. Thus, in some embodiments, the kit further comprises antibodies. In some embodiments, the antibodies are specific for surface antigens of the microorganism or the sample comprising cells.

In some embodiments, the analysis is high-throughput sequencing. In some embodiments, the high-throughput sequencing is metagenomic sequencing. Thus, in some embodiments, the kit further comprises materials for DNA extraction, primers, or a combination thereof.

In some embodiments, the kit also provides measuring accumulation in the microorganisms or sample comprising cells. Thus, in some embodiments, the kit further comprises materials for DNA extraction, primers, or a combination thereof.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Consideration of a Liquid Mutation-Accumulation Experiment to Measure Mutation Rates by Successive Serial Dilution

In addition to the concerns relating to MA experiments mentioned above, depending on how a researcher picks and spreads their colonies, edge effects may skew cell division estimates, as some parts of a colony experience more divisions than others (M. R. Warren, et al., eLife, (2019) 8:e41093). Though single-cell bottlenecks are expected to overwhelm any but the most stringent selection (lethality), the harmonic mean effective population size is closer to 14 than 1 (L. M. Wahl et al., Evolution, (2022) 76(3):528-540; A. Mahilkar, et al., Scientific Reports, (2022) 12(1):15470). Several authors propose selection is a significant factor in a generic MA experiment, either throughout or in at least the first two weeks (L. Bosshard, et al., BMC genomics, (2020) 21(1):253). Colony choice instigated by researchers during serial bottlenecking is also at risk of researcher bias, in that humans tend to pick the colonies they can see, rather than just any colony. Humans can also select, consciously or without realizing, a certain colony phenotype, perhaps skewing the distribution of mutations observed in an experiment. The sum of these criticisms is not entirely without merit; there is certainly some selection, positive and purifying, occurring in a MA experiment (T. T. Kibota, et al., Nature, (1996) 381(6584):694-696; W. Sung, et al., Molecular Biology and Evolution, (2015) 32(7):1672-1683). Regardless, it is difficult to expect mutation-rate estimations are off by a factor of 2; for this to be the case, half of all mutations would have to be lethal.

Some of the above concerns of bias may be addressed by a MA experiment with a less structured environment. Researchers have long appreciated the ability of serial dilution to reach countable sub-samples of bacterial populations. In principle, some level of dilution can be achieved so that half of all wells inoculated by a dilution contain a viable cell, and half do not. By reaching this level of dilution or lower, relatively high confidence can be maintained that a single viable bacterium was the source of the growth observed in a well 24 hours later. Moreover, wild-type E. coli can reach carrying capacity within 12-14 hours, given a cell division rate of 20-30 minutes (A. R. Tuttle, et al., Current Protocols, (2021) 1(1):e20). This suggests that a fixed fitness burden of up to 50 percent may reasonably be tolerated without affecting our ability to select it. If carrying capacity remains relatively constant, the number of cell divisions should be quite easy to determine relative to a plate MA, which typically develops wildly different colony sizes over time (H. Lee, et al., Proceedings of the National Academy of Sciences of the United States of America, (2012) 109(41):E2774-2783). The liquid-based single-cell bottleneck eschews the semi-structured agar environment, and reduces the potential risk of artificial selection imposed by researchers. It is also readily recognizable that a liquid MA lends itself to automation by liquid handling robots, in contrast to the agar-based MA's that escape easy conversion to robotic control. If this automation could be achieved, one of the most laborious aspects of obtaining mutation rates by an MA experiment could be made more tolerable.

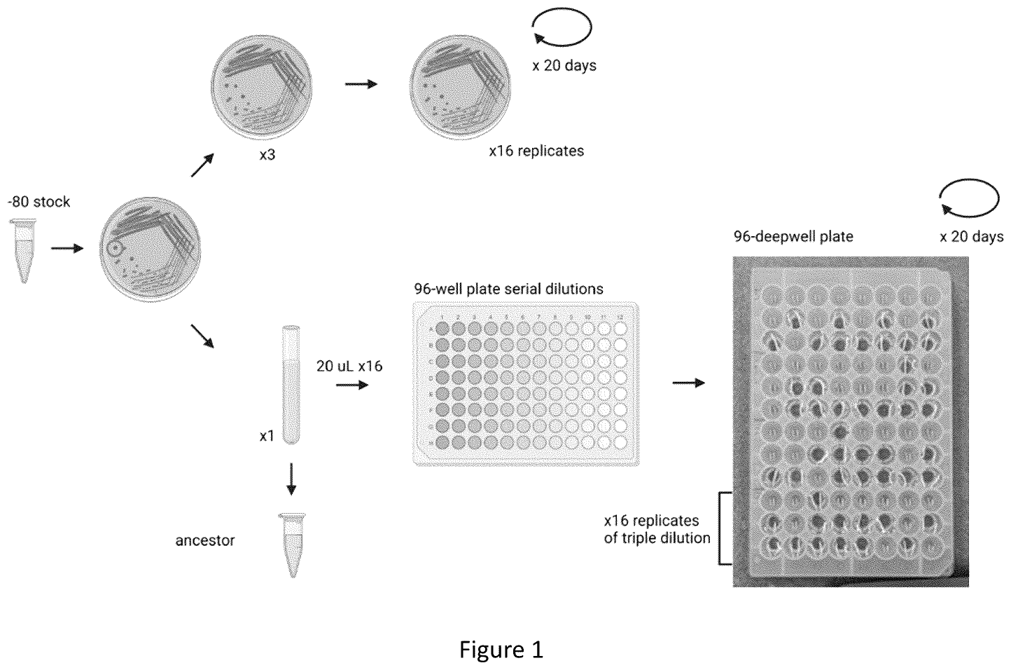

To compare and contrast the output of MA experiments run by liquid serial dilution or by standard agar plates, a well-studied E. coli strain, MutL- also known as mismatch-repair deficient (MMR-), K12, MG1655, was used. This strain has already been characterized by a standard MA experiment, at least twice (H. Lee, et al., Proceedings of the National Academy of Sciences of the United States of America, (2012) 109(41):E2774-2783; W. Wei, et al., Nature Communications, (2022) 13(1):4752). This strain is particularly useful because it features a distinct mutation spectrum, as well as an elevated mutation rate, at least 100-fold higher than WT. The elevated mutation rate allows for a temporally shorter MA with relatively few samples to yield an abundance of mutations, along with measurable phenotypes. A plate and a liquid MA were ran in tandem from the same starting single colony for 20 days (FIG. 1), and the DNA was sequenced afterwards. A modest difference in the mutation spectrum of E. coli was found, but moreso, the relative invariance of mutation-rates estimated from the experiment between growth types, 3.10×10⁻⁸ vs. 2.80×10⁻⁸ base-pair substitutions (BPS)/site/generation was recognized for plate and liquid MA's, respectively. These data suggest the liquid MA is a compelling option for identifying the mutation rates for micro-organisms which do not grow well on agar surfaces.

The results are described herein.

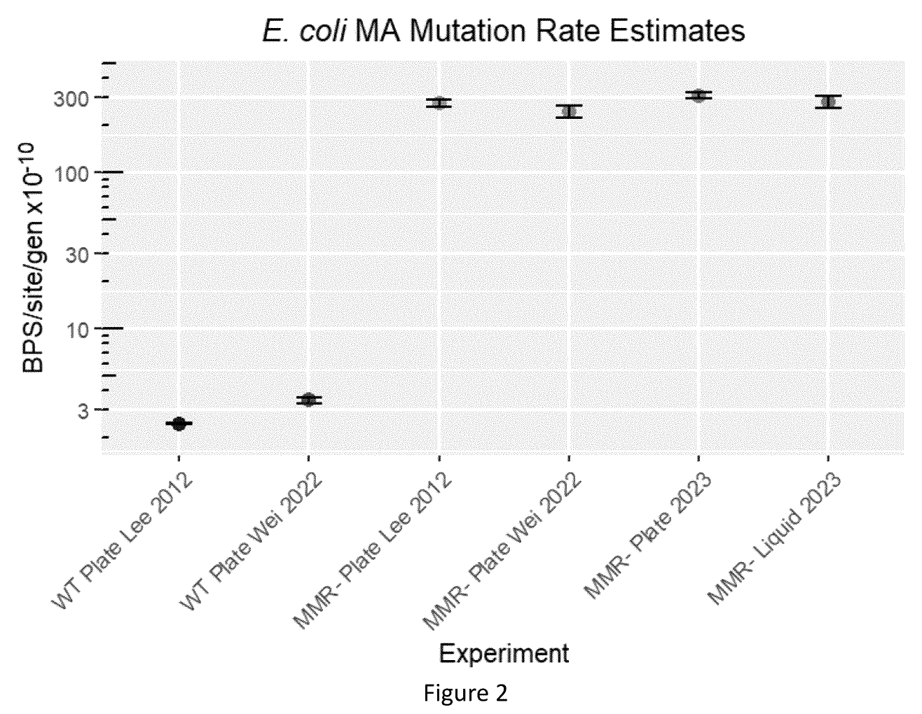

The tandem mutation-accumulation experiments in liquid and upon plates of otherwise identical nutrient content are quite nearly identical in estimation of mutation rate per site per generation and in general agreement with previous mutation-accumulation experiments (FIG. 2). This resulted in similar mutation burdens over the course of the experiment (Table 1), with the difference in burden being explained by the liquid MA experiencing slightly more cell divisions per day. Specifically, the liquid MA lines had a higher mutation burden in base pair substitutions (BPS) by the end of the experiment, almost 10%, which can be explained by the cells having experienced 10% more cell divisions (29.9 vs. 26.9 mean cell divisions per line per day). Also in concurrence with previous MA experiments (H. Lee, et al., Proceedings of the National Academy of Sciences of the United States of America, (2012) 109(41):E2774-2783), the indel rate was found to be reduced relative to base-pair substitution rates (Table 1).

TABLE 1
Summary Statistics for the Liquid and Plate MA experiments.

| | | | | | Mutation | |
|---|---|---|---|---|---|---|
| | | Mean BPS | Mean | Mean | BPS/site/ | Indel/site/ |
| | Sample | per line | INDEL | generations | gen ×10⁻⁸ | gen ×10−⁸ |
| Treatment | Size | (S.E.M.) | per line | per transfer | (S.E.M.) | (S.E.M.) |
| Liquid | 16 | 76.8 (+/−6.32) | 17.8 (+/−1.44) | 29.9 | 2.80 (+/−0.24) | 0.65 (+/−0.053) |
| Plate | 15 | 76.7 (+/−4.28) | 12.3 (+/−0.78) | 26.9 | 3.10 (+/−0.17) | 0.49 (+/−0.031) |

S.E.M.: Standard Error of the Mean.

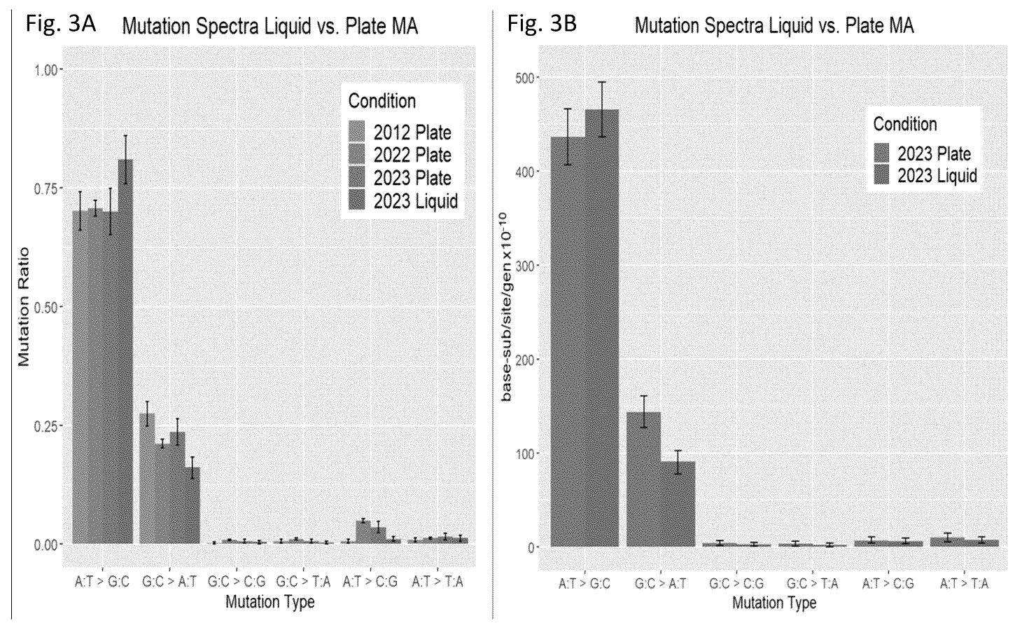

The mutation spectra are similar to previous estimates from 2012 and 2022 (FIG. 3A), although a χ-square test detected a significant difference between liquid and plate spectra (FIG. 3B) (p=8.8×e-8), driven by both a reduction in G:C→A:T transitions and an increase in A:T→G:C transitions (Table 2). Though the difference by ratio is modest in appearance, the net effect over 20 days yields a difference of 74 G:C→A:T mutations across summed across the 16 lines (Table 3), a 37% increase in occurrence in the plate vs. liquid MA experiments.

TABLE 2
Raw Data of the Mutation Spectrum

| Mutation | L Total | | P Total | |
|---|---|---|---|---|
| Type | Count | L Frequency | Count | P Frequency |
| A:T→G:C | 995 | 0.81 | 806 | 0.71 |
| G:C→A:T | 198 | 0.16 | 272 | 0.236 |
| A:T→C:G | 12 | 0.010 | 13 | 0.011 |
| A:T→T:A | 15 | 0.012 | 18 | 0.016 |
| G:C→T:A | 5 | 0.004 | 6 | 0.005 |
| G:C→C:G | 4 | 0.003 | 7 | 0.006 |

TABLE 3
Base-Pair Substitution Rate Per Line

| Line | indel | snps | gen | bps rate/site/gen | indel rate/site/gen | |
|---|---|---|---|---|---|---|
| L01: | 24 | 86 | 29.9 | 3.10E−08 | 8.65E−09 | |
| L02: | 17 | 81 | 29.9 | 2.92E−08 | 6.12E−09 | |
| L03: | 15 | 63 | 29.9 | 2.27E−08 | 5.40E−09 | |
| L04: | 5 | 60 | 29.9 | 2.16E−08 | 1.80E−09 | |
| L05: | 23 | 96 | 29.9 | 3.46E−08 | 8.29E−09 | |
| L06: | 28 | 155 | 29.9 | 5.58E−08 | 1.01E−08 | |
| L07: | 21 | 79 | 29.9 | 2.85E−08 | 7.57E−09 | |
| L08: | 13 | 50 | 29.9 | 1.80E−08 | 4.68E−09 | |
| L09: | 17 | 53 | 29.9 | 1.91E−08 | 6.12E−09 | |
| L10: | 20 | 75 | 29.9 | 2.70E−08 | 7.21E−09 | |
| L11: | 19 | 80 | 29.9 | 2.88E−08 | 6.85E−09 | |
| L12: | 21 | 77 | 29.9 | 2.77E−08 | 7.57E−09 | |
| L13: | 20 | 68 | 29.9 | 2.45E−08 | 7.21E−09 | |
| L14: | 15 | 44 | 29.9 | 1.59E−08 | 5.40E−09 | |
| L15: | 8 | 77 | 29.9 | 2.77E−08 | 2.88E−09 | |
| L16: | 18 | 85 | 29.9 | 3.06E−08 | 6.48E−09 | |
| P25: | 9 | 86 | 26.9 | 3.44E−08 | 3.60E−09 | |
| P26: | 14 | 65 | 26.9 | 2.60E−08 | 5.61E−09 | |
| P27: | 14 | 87 | 26.9 | 3.48E−08 | 5.61E−09 | |
| P28: | 15 | 97 | 26.9 | 3.88E−08 | 6.01E−09 | |
| P29: | 10 | 65 | 26.9 | 2.60E−08 | 4.00E−09 | |
| P30: | 15 | 87 | 26.9 | 3.48E−08 | 6.01E−09 | |
| P31: | 8 | 88 | 26.9 | 3.52E−08 | 3.20E−09 | |
| P32: | 10 | 50 | 26.9 | 2.00E−08 | 4.00E−09 | |
| P33: | 14 | 90 | 26.9 | 3.60E−08 | 5.61E−09 | |
| P34: | 13 | 60 | 26.9 | 2.40E−08 | 5.21E−09 | |
| P35: | 15 | 106 | 26.9 | 4.24E−08 | 6.01E−09 | |
| P36: | 10 | 63 | 26.9 | 2.52E−08 | 4.00E−09 | |
| P37: | 0 | 0 | 26.9 | 0.00E+00 | 0.00E+00 | |
| P38: | 7 | 54 | 26.9 | 2.16E−08 | 2.80E−09 | |
| P39: | 13 | 76 | 26.9 | 3.04E−08 | 5.21E−09 | |
| P40: | 17 | 76 | 26.9 | 3.04E−08 | 6.81E−09 | |
| | | | | 9.11E−09 | 2.08E−09 | standard deviation L |
| | | | | 1.00E−08 | 1.69E−09 | standard deviation P |

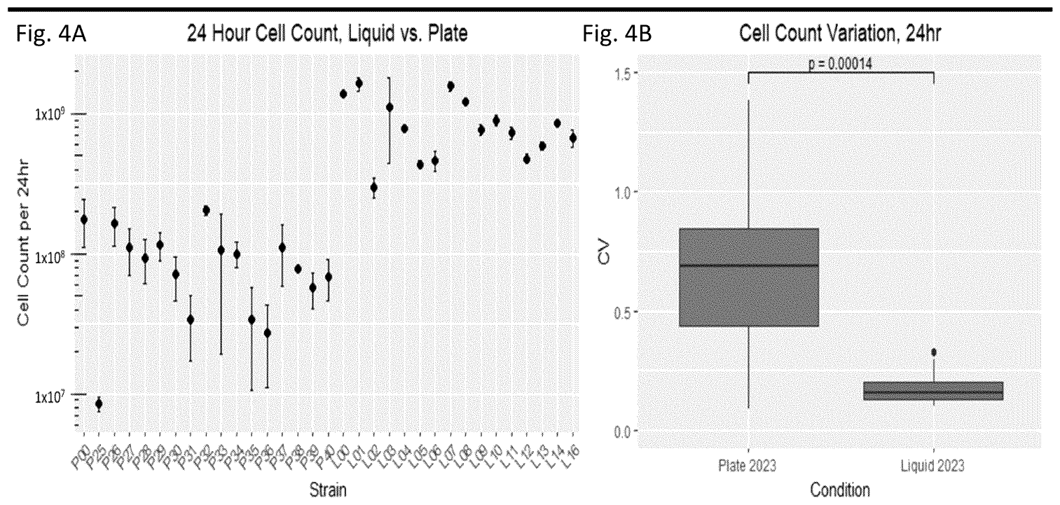

The liquid MA experienced roughly 3 additional mutations per day (29.9 vs 26.9, p value=7.662e-11, t-test), a consequence of the 1 mL growth volume allotted per well in the 96-deepwell plate. A difference in the number of cell divisions is noted not only in count per day but also in variation per colony, which is readily discerned in a comparison of the standard error of the mean in the estimates of number of cells per day (FIG. 4). The increased variance, readily discernable by eye in FIG. 4A, error bars liquid samples vs. plate samples, can be compared by calculating the coefficient of variation (CV), which normalizes the standard deviation of a sample by its mean value. The difference in CV is statistically significant (FIG. 4B, p value=0.00014, t-test), and is readily explained by colony dynamics. When colonies are close to one another they experience nutrient competition and grow less quickly. Random differences in spacing between colonies leads to variation in colony size, and thus increased variation in the total number of cell divisions experienced in a day relative to liquid cultures. It is also true that colonies shrink on average over the course over a mutation-accumulation experiment, as mutations accumulate and fitness degrades. This shrinking colony size is readily seen in measurements of fitness, for example in plate line 25 (FIG. 4A).

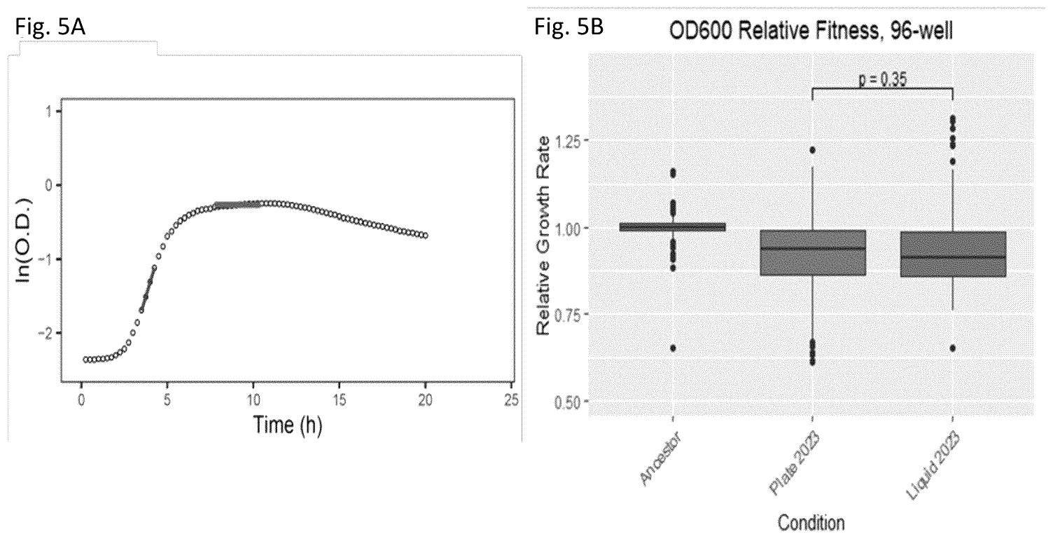

One hallmark of a successful MA experiment is a decline in mean fitness of the lines (T. T. Kibota, et al., Nature, (1996) 381(6584):694-696). To ensure the MA ran as anticipated, fitness was measured through use of a 96-well plate reader, which determines changes in optical-density over time. In particular, the maximum slope of optical-density change in the first 8 hours of the experiment is sufficient to provide a useful proxy for maximum cell growth rate (FIG. 5A and FIG. 5B), as has been employed elsewhere. The MA lines tended to have higher optical-density than the ancestor by 10 hours or so, and the OD readings peaked around 12 hours and seemed to recede some 20% by the end of the growth experiment (FIG. 5A). Further, overall, the MA lines had higher OD readings toward the tail of the experiment, relative to the ancestor (FIG. 5C). It is possible that the MA lines, in particular the liquid MA lines, had evolved higher carrying capacity by some form of selection. To verify the results from the 96-well plate reader, CFU's of the 12-hour time-point was ran (FIG. 5D). Despite the optical-density being statistically significantly higher in the MA lines vs. the ancestor, this OD600 absorbance was not due to increased numbers of cells. Thus, it was concluded that the OD600 measurement by plate reader of carrying capacity is of limited use, at least in MA lines of hypermutator E. coli. Based on the growth phase and CFU counts (FIG. 5B and FIG. 5D), the average fitness cost per mutation was calculated to be 0.001.

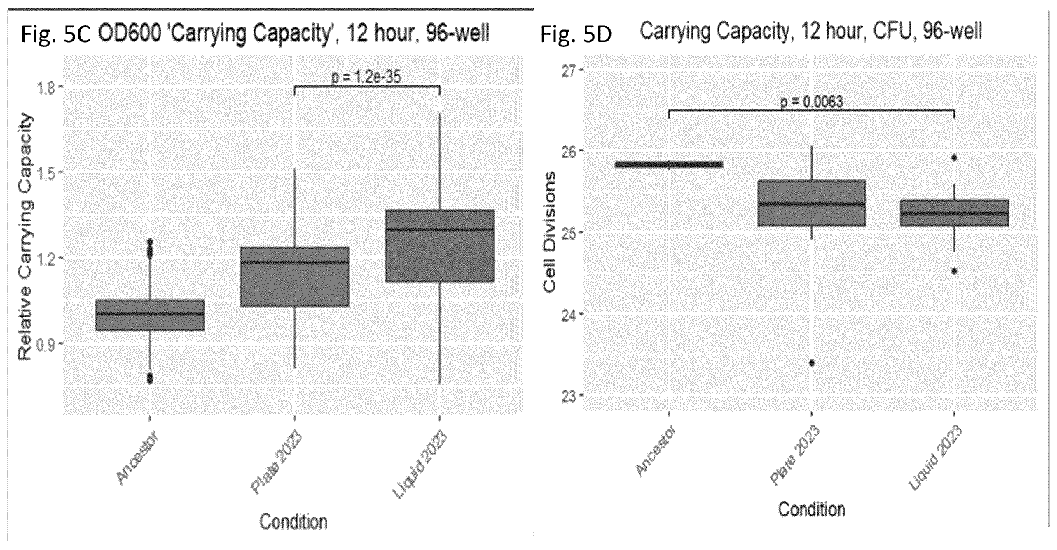

Through the course of an MA, a distribution of fitness decline is expected and was observed, with an average fitness cost of approximately 0.001 per mutation. On day 15 of the liquid MA experiment, one particular line, Liquid line 2 (L02), was perceived to have significantly decreased in 24-hour cell count on the day of transfer, moreso than all others (FIG. 6A). Time-points of day 14, 15, and 16 were collected for the line, in the hopes of determining which mutation was most responsible for the loss in cell count. Given the mutation rate, number of cell divisions, and genome size, 4 mutations were expected to accumulate on average per line per day. Four new mutations were detected between days 14 and 15 (FIG. 6B), a result of the bottlenecking process. Two of the mutations are synonymous, and one is intergenic in the middle of an operon, and thus seem unlikely to be responsible for such dramatic phenotypic decline. The 4th mutation, in the CadA gene, appears quite promising, as it has been described as useful to elongate the duration of cell growth in nutrient restricted environments (P. L. Moreau, Journal of Bacteriology, (2007) 189(6):2249-2261).

In support of previous experimental results, the degree of selection within the MA was found to be significant though modest in nature. If mutations accumulate in an entirely neutral process, a ratio of 5.74 coding mutations per every 1 non-coding mutation would be expected, a reflection of the ratio of coding to non-coding DNA in E. coli MG1655 (H. Lee, et al., Proceedings of the National Academy of Sciences of the United States of America, (2012) 109(41):E2774-2783), or 3.95×10⁶ nucleotides coding to 6.88×10⁵ non-coding nucleotides. The occurrence of indels and base-subs at intergenic (between genes) or intragenic (within genes) nucleotide site is expected to be somewhat lower than the neutral expectation, in account of the advent of lethal mutations, with a stronger effect on indels due to their more disruptive character. It was found that the ratio of intergenic/intragenic indels is 2.68:1 in the liquid MA and 2.54 in the plate MA, significantly different from the expected ratio of 5.74:1 (1.15e-28, χ-square). Further, for base-subs, it was found that the intragenic/intergenic ratio was 9.91:1 (p-value 2.62e-74, χ-square) and 7.71:1, for liquid and plate MA's, respectively. These results indicate the evidence of purifying selection in the case of indels (fewer indels in coding sequences relative to null expectation), and positive selection for base substitutions in coding sequences. These trends are in line with recent findings of selection within MA experiments (L. M. Wahl et al., Evolution, (2022) 76(3):528-540; A. Mahilkar, et al., Scientific Reports, (2022) 12(1):15470), particularly are built into the experimental design, as was the case of the liquid MA.

Within coding nucleotides, the expected ratio of non-synonymous to synonymous mutations is roughly 3.10, corresponding to 2942979 effectively non-synonymous sites to 947643 effectively synonymous sites. Compared to the expected ratio, a ratio of 2.13 was detected in the liquid MA and 2.07 for the Plate MA. Both deviate from the null expectation (χ² p=2.27×10⁻²¹), but are not significantly different from one another. These results are nearly identical to those reported in 2012 (H. Lee, et al., Proceedings of the National Academy of Sciences of the United States of America, (2012) 109(41):E2774-2783). The difference in ratios suggest an excess of synonymous mutations or a dearth of non-synonymous mutations compared to the null expectation in MMR—E. coli. This is additionally in line with recent commentary on bacterial MA experiments (L. M. Wahl et al., Evolution, (2022) 76(3):528-540; A. Mahilkar, et al., Scientific Reports, (2022) 12(1):15470).

With regard to the comparison, the Liquid MA appears to yield comparable results to the standard plate MA. It is possible that, with selection likely reducing the ratio of non-synonymous mutations from 3.1:1 to 2.1:1, the true mutation rate of MMR—E. coli may be roughly a third higher than is reported here and in previous experiments, or 3.8×10⁻⁸ base pair substitutions per site per generation.

In summary, the liquid MA largely recapitulated the findings of a plate MA for MMR—E. coli, with several modest differences. This result supports the perspective that MA experiments, even those which have been run on agar plates, provide reasonable estimates of organism mutation rates. It would appear that E. coli is not significantly perturbed in mutation rate by growing either in liquid or on plate colonies, over the course of 24 hours. Despite some fraction of the cells being nutrient deprived for 6-14 hours (M. R. Warren, et al., eLife, (2019) 8:e41093), E. coli appears evolved to manage this environmental challenge with no significant change to its genome-wide mutation rate. Given the expectation that bacteria routinely encounter nutrient deprivation for hours, or perhaps even days or months in their evolutionary past, this result is not exceptional.

A primary finding of this research is that the liquid MA, single-cell bottlenecking by serial dilution does indeed work. The strength of the liquid MA lies in its scalability, in the promise of automation, which in principle requires no more than picking which well a researcher would like to load per sample into a robot for serial dilution. In the hands of a robot, contamination will be less of a risk while running MA experiments. Given that some organisms do not grow well on agar plates, this protocol may also provide some researchers with an avenue to pursue questions of variation in mutation rate which have previously been intractable.

It is noted also that there is overall resilience of the standard MA in the face of some criticisms to the technique over the years. Despite fear of induced selection or bias on behalf of the colony picker, cursory examination of MA results between liquid and plate demonstrate the reliability of both protocols. A large difference in mutation rate was not detected, in mean fitness cost per mutation, or in DN/DS ratio. One minor benefit of running a liquid MA is a less variant number of cell divisions per day per line. This may result in a more accurate estimation of mutation rates, though if true the effect is modest, if present at all: a difference of 2% was detected between the estimates of mutation rate between liquid and plate MA's.

It is noted also that some plate MMR—MA lines have produced mutation spectra of similar (Foster et al., Genetics (2018) 209(4):1029-1042) skew to those reported here. With this preface, it is noted that the liquid mutation rate is skewed even more strongly to A:T→G:C than the previous results of plate MA experiments. The most parsimonious mechanism for this mutation spectrum of transitions is base-pair tautomerization, as proposed by Watson and Crick in 1953 (J. D. Watson, et al., Cold Spring Harbor Symposia on Quantitative Biology, (1953) 18:123-131) and iterated upon since (L. Slocombe, et al., Phys. Chem. Chem. Phys., (2021) 23(7):4141-4150). Watson and Crick noted that the temporary shift of hydrogen atoms to neighboring atoms might cause some level of nucleotide mispairing. They hypothesized that the A:T→G:C mutation ought to be more frequent than the G:C→A:T, because the former requires a single hydrogen atom shift, while the latter requires atom shifts. It is unclear if or why the medium of growth would alter the ratio of tautomerization, though it is known that the spent culture of E. coli significantly changes in pH (increase to pH of 9) and chemical composition (M. G. Behringer, et al., Proceedings of the National Academy of Sciences, (2018) 115(20):E4642-E4650; G. Sezonov, et al., Journal of Bacteriology, (2007) 189(23):8746-8749) which also affects gene expression profiles. Further, the decreased G:C→A:T rate may be partly explained by the E. coli of the liquid MA being exposed to less oxidative stress resulting in fewer damaged guanosine residues, a base prone to oxidative damage (K. Kino, et al., Genes and Environment, (2017) 39(1):21; J. Y. Hahm, et al., Experimental & Molecular Medicine, (2022) 54(10):1626-1642). However, growth of wild-type E. coli in the complete absence of oxygen mildly increased mutation rates, on the order of 2-fold, with no reduction in mutations to guanosines (S. Shewaramani, et al., PLOS Genetics, (2017) 13(1):1-22). Regardless, the recurrent theme of MMR—E. coli and its derived strains are that the mutation spectrum is skewed in favor of transitions, as expected (H. Lee, et al., Proceedings of the National Academy of Sciences of the United States of America, (2012) 109(41):E2774-2783; W. Wei, et al., Nature Communications, (2022) 13(1):4752). The data of this work and prior MMR—growth experiments lend credence to the idea that tautomerization, if rare, is likely biologically relevant, such that life evolves DNA-repair pathways to manage its occurrence. It is noted that the degree of the imbalance in mutation types is potentially quite useful in the verification of novel sequencing technologies seeking to detect DNA mutations, to ensure that they are working properly.

With regard to fitness estimates of E. coli, discrepancies between the 96-well plate reader OD and actual cell count is a known issue (A. Rana, et al., Genetics, (2021) 219(2):117), but reiteration will hopefully dissuade researchers from relying on 96-well plate readers overmuch, in the context of mutation-accumulation experiments. As to why the discrepancy exists, at least in this experiment, it is proposed that the mutation-burdened cells of a MA experiment are more prone to biofilm formation or flocculating, sinking to the bottom of a well containing liquid growth medium. Either phenotype results in a layering of cells, which may lead to increased occlusion of the OD detector and yielding unusually high OD readings. Regardless of the ultimate cause of the outlier readings, the conclusion remains that attempting to quantify relative carrying capacity from OD600 in plate readers from cells with many accumulated mutations is at best done with caution, and should be backed up with CFU measurements.

A retrospective of the experiment suggests potential avenues for improved efficiency. The experimental design was strongly influenced by a desire for relative surety of obtaining single-cell bottlenecks. Alternative strategies exist, for example one in which serial dilutions are performed by doing 1:2 dilutions from a starting 10⁻⁶ dilution. Within eight serial dilutions the dilution should be complete, and yield wells which have no growth inside them. A researcher would then simply select the last serial dilution on with a grown bacterial culture. This strategy would yield strong bottlenecks, which would allow a fair approximation of mutation rate with higher throughput. Instead of 4 samples per 96-deepwell plate, the yield would become 12. The dilution strategy of the deepwell plate could also make use of a multichannel pipettor; the protocol described in this experiment did not, when inoculating each row of serial dilution. There is some risk of increased selection in the experiment, but the protocol would be perhaps 2-3× faster with a 3× increase in yield.

The methods are described herein.

Frozen stocks of E. coli MMR—were thawed and streaked upon an LB plate (LB Agar, Miller). 24 hours later, a single colony was chosen, from which 1 10 mL liquid LB tube (LB Broth, Miller) was inoculated while 4 additional LB plates were streaked. From the four agar plates, 40 colonies were chosen to be the set of 40 MA lines, and comprised the first day of the plate MA. For each of these 40 lines, per day, every 24 hours, a single colony was selected and streaked on half of a LB plate, with two or three streaks to obtain single colonies. The first streak was a toothpick line (autoclaved toothpicks), and one or two consecutive streaks thereafter came from autoclaved wooden sticks. The last colony of a streak was always chosen for transfer, unless forced by poor streaking to pick the last convincing single colony. Plates were loaded into a 37° C. incubator and grown overnight for 24 hours, +/−2 hours, before streaking was repeated on new LB agar plates the next day.

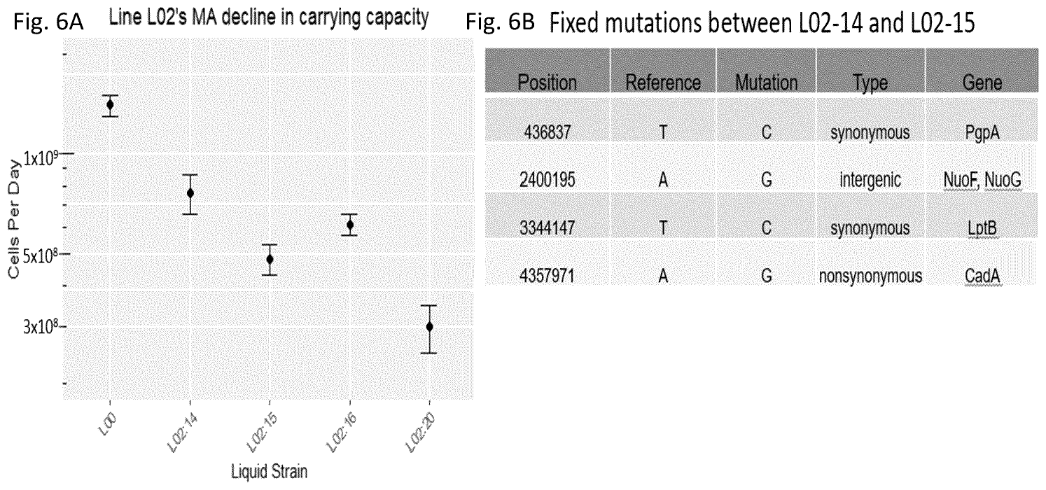

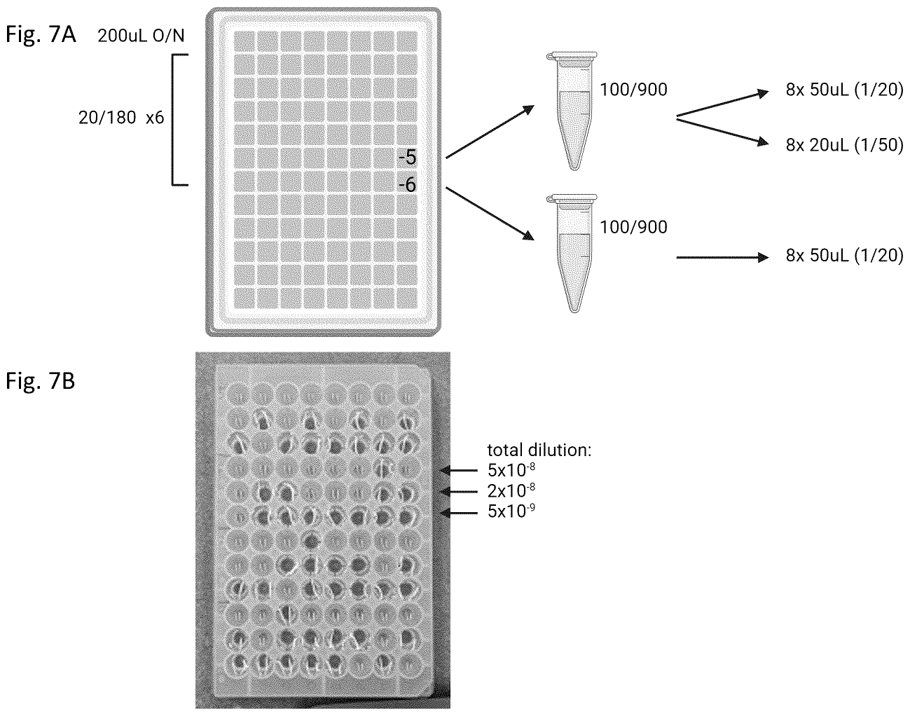

From the 10 mL liquid LB tube, first an ancestor line frozen stock was made and then 16 wells of 20 μL were taken and run through serial dilution (FIG. 1). Briefly, E. coli were serially diluted to the −5 and −6 wells by standard methods and multichannel pipettors in 180 μL 1×PBS in 96 well plates (FIG. 7). Then, 100 μL of the −5 was added to 900 μL of 1×PBS to create a “−7” dilution of 1 mL per sample, and the same was done to the −6 well to create a “−8” dilution of 1 mL per sample. From the −7 dilution, 20 μL was inoculated to a single row of a 96-deepwell plate holding 950 μL of liquid LB, 8 wells. From the −8 dilution, a second row for the same sample was loaded with 50 μL, and for a 3rd row the −8 dilution was again used to inoculate 20 μL. This results in an order of magnitude scale of dilution in the 96 deepwell plate, where each liquid sample comprises now 24 replicates of varying serial dilution. On a 96-deepwell plate four samples were arrayed, for a total of 4 96-deepwell plates, or 16 replicates, per transfer day. After 24 hours, some of the serial dilution wells have turbid growth, while others remain clear. At the lower serial dilutions, a majority of the wells should be clear. As a rule, the turbid well from the lowest serial dilution would be chosen for the next round of serial dilution. In the event of a tie, the one on the left-hand side was chosen. After 15 days, frozen stocks were made of both the liquid and the plate MA. After noticing an apparent decrease in carrying capacity in one of the liquid MA lines, the experiment was stopped at 20 days for characterization and sequencing. Based on the previous MA experiment (H. Lee, et al., Proceedings of the National Academy of Sciences of the United States of America, (2012) 109(41):E2774-2783), it was expected 20 days would result in a sufficient number of mutations to analyze the data.

After the MA's were complete and frozen stocks were obtained, DNA was extracted by first growing the frozen stocks overnight in 10 mL tubes and then running the Promega Wizard DNA Extraction Kit, with minor modifications immaterial to results save by increasing yield to greater than 1 μg per sample. Genomic DNA was quantified, and submitted to the Beijing Genomics Institute for their in house library prep and DNB-sequencing.

Raw sequence reads were analyzed by fastqc to confirm a successful sequencing run. Reads were then filtered with Trimmomatic to remove adapter sequences. Reads were then aligned to the E. coli K12 reference genome (ncbi.nlm.nih(dot)gov/nuccore/U00096.2) with BWA, the Burrows-Wheeler Aligner. After alignment and conversion to bam files by SamTools, the mutation caller GATK2 generated VCF files of novel mutations when comparing the ancestor to the evolved lines. Output VCF files were additionally annotated by SnpEff to determine Dn/Ds and Genic/Intergenic ratios. The results of the mutation rate and mutation spectrum were graphed with ggplot2 in R. Error bars for the mutation spectrum reflect the poisson 95% confidence intervals, similar to previous studies, which were calculated using the Epitools package for R with pois.approx.

To estimate the number of cell divisions that occurred over the course of the 20-day experiment, the liquid and plate MAs were handled differently. For the plate MA, 20-day frozen stocks and the ancestor were thawed and grown in LB overnight in 10 mL tubes. This liquid was then streaked on LB plates. After 24 hours, the colonies were resuspended in LB and serially diluted to the −6 plate, and plated to count colony forming units (CFU's) per mL. An average of the entire set of 16 plate MA samples was used to calculate mean number of cell divisions per day at day 20, the ancestor calculated mean number of cell divisions per day at day 1. The average between the day 1 and mean of day 20 samples was used as an estimate for number of cell divisions per line.

To estimate cell divisions from the liquid MA, 20-day frozen stocks and the ancestor were thawed and grown overnight in LB in 10 mL tubes. After 24 hours, 20 μL aliquots were taken from the overnight cultures and serially diluted down to single cell bottlenecks, before being grown up in 96-deepwell plates, in a method identical to the MA serial dilution. After an additional 24 hours, 4 turbid replicates were chosen for serial dilution to count CFUs. From this, the number of cell divisions per day was calculated, at day 1 and day 20. The mean of day 20 cell division estimates, and the average between the day 1 number of cell divisions and mean of day 20 lines was used as an estimate for average total number of cell divisions during the experiment.

To phenotype the lines for fitness and carrying capacity using a plate reader, a 96-well plate was used. Two lines and an ancestor were thawed out per day and grown overnight. 100 μL of overnight culture was then added to 10 mL of LB in 15 mL tubes. These 15 mL tubes were inverted 15 times to mix. The mixtures were then loaded onto a 96-well plate, 200 μL per plate, staggered such that rows 1, 4, 7, and 10 received the ancestor, 2, 5, 8, 11 received MA line 1, and 3, 6, 9, 12 received MA plate was double-sealed with parafilm and loaded into a plate reader for 24 hours of optical-density analysis, at 37° C., with readings taken at a wavelength of 600 nm (OD600). Growth rate was obtained by calculating the largest slope of growth between 2 and 10 hours, when averaged across 5 time points. Carrying capacity was calculated at average optical-density at 12 hours, averaged over 5 time points. Data were normalized per day in comparison to the ancestor, and then the relative fitness and relative “carrying capacity” were graphed in R.

To phenotype the lines for carrying capacity after 12 hours by CFU, all lines were thawed in 10 mL tubes of LB overnight. The lines were then diluted in quadruplicate to 1/100th concentration in a 96-well plate, by adding 2 μL of mixed overnight culture to 198 μL of fresh LB. These plates were then sealed with parafilm in 2 layers and loaded into a 37 degree incushaker shaking at 200 RPM, secured by magnets, for 12 hours. After 12 hours, the E. coli were serial diluted to the −7 plate and CFUs were obtained by spreading 100 μL on LB plates. Data were processed by collecting CFU estimates, running t tests, and graphing the different CFU averages with standard error of the mean in R.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of culturing microorganisms or sample comprising cells comprising inoculating and incubating a microorganism or a sample comprising cells in a liquid culture system, wherein the liquid culture system comprises a vessel and a liquid medium.

2. The method of claim 1, wherein the microorganism is one or more selected from the group consisting of bacteria, fungi, and yeast.

3. The method of claim 1, wherein the cell is a cancerous cell, a diseased cell, or an atypical cell.

4. The method of claim 3, wherein the cell is a HEK293 cell.

5. The method of claim 2, wherein the microorganism is bacteria, wherein the bacteria is (a) from the genus synechocystis or selected from the group consisting of: E. coli, Bacillus subtilis, Caulobacter crescentus, Mycobacterium tuberculosis, Streptococcus pneumoniae, Saphylococcus aureus, Listeria monocytogenes, Campylobacter, Yersinia pestis, Chlamydia trachomatis, Vibrio cholerae, and others; or (b) wherein the microorganism is selected from the group consisting of Thermus aquaticus and Sulfolobus acidocaldarius.

6. The method of claim 1, wherein the microorganism or sample comprising cells is a mismatch repair deficient.

7. The method of claim 1, wherein the liquid medium is one or more selected from the group consisting of Luria Bertani (LB) broth, Terrific Broth (TB), M9 minimal media, Sabouraud Dextrose (SD) broth, Yeast medium (YM) broth, (Ymin) yeast synthetic minimal media, BG-11 culture medium, a modified BG-11 culture medium, f/2 culture medium, a modified f/2 culture medium, ocean water, lake water, river water, wastewater, and other available water sources.

8. The method of claim 1, wherein the method further comprises at least one step of diluting the microorganism or sample comprising cells.

9. The method of claim 8, wherein the microorganism or sample comprising cells is diluted at least twice, is diluted until a vessel comprises a single cell, or a combination thereof.

10. The method of claim 9, wherein the vessel comprising a single cell is the vessel with the smallest turbidity, smallest color change, or a combination thereof.

11. The method of claim 8, wherein the step of diluting the microorganism or sample comprising cells is automated.

12. The method of claim 1, wherein the microorganism or sample comprising cells is incubated at a temperature of about 25° C. to about 70° C.

13. The method of claim 1, wherein the medium has a pH of 3 to 10.

14. The method of claim 1, further comprising an analysis step.

15. The method of claim 14, wherein the analysis step is selected from the group consisting of: plating the microorganism or the sample comprising cells on a media, differential staining, antibody staining, and high-throughput sequencing.

16. The method of claim 15, wherein the media comprises permissive growth conditions or inhibitory growth conditions; wherein the differential staining is Gram staining; wherein the antibody staining is ELISA; or wherein the high-throughput sequencing is metagenomic sequencing.

17. An assay system for measuring mutation accumulation in a microorganism or a sample comprising cells comprising the method of claim 1 and a DNA analysis step.

18. The assay system for measuring mutation accumulation in a microorganism or a sample comprising cells of claim 17, wherein the DNA analysis step is DNA sequencing.

19. An assay system for detecting the presence of a microorganism or a sample comprising cells in a sample comprising the method of claim 1.

20. The assay system of claim 19, wherein the assay system is used to detect urinary tract infection.
